(19) United States
(12) Patent Application Publication
(10) Pub. No.: US 2020/0360423 A1
(43) Pub. Date: Nov. 19, 2020

(54) SOLID CONCENTRATED CONSTIPATION TREATMENT FORMULATIONS

(71) Applicant: ColonaryConcepts LLC, Palm Beach Gardens, FL (US)

(72) Inventors: Michael K. ALLIO, Palm Beach Gardens, FL (US); Corey A. SIEGEL, Palm Beach Gardens, FL (US); Joshua KORZENIK, Palm Beach Gardens, FL (US)

(21) Appl. No.: 16/961,904

(22) PCT Filed: Jan. 11, 2019
(86) PCT No.: PCT/US2019/013368

§ 371 Date: Jul. 13, 2020

Related U.S. Application Data
(60) Provisional application No. 62/617,061, filed on Jan. 12, 2018.

(51) Int. Cl. A61K 31/765; A61K 9/00

(52) U.S. Cl. CPC A61K 31/765; A61K 9/0056

(57) ABSTRACT

Described herein are compositions and methods for treating constipation in an individual. In some embodiments, the composition and method for treating constipation comprises providing an individual with a composition that comprises a food item comprising one or more ingredients and a concentrated amount of laxative.

### CROSS-REFERENCE

This application claims the benefit of U.S. Provisional Application No. 62/617,061 filed Jan. 12, 2018 which application is incorporated herein by reference.

### BACKGROUND

Constipation is a widespread condition worldwide. Constipation is associated with flatus and intestinal distention which cause discomfort and abdominal pain to sufferers.

There are numerous causes of constipation including medical conditions and iatrogenic constipation resulting from certain prescribed medications.

### SUMMARY

Constipation affects millions of people yearly worldwide. The condition is attributed to numerous causes including certain medical conditions and physiological conditions such as Irritable Bowel Syndrome, GI motility disorders, pregnancy, sedentary lifestyle, and diet. In addition numerous medications are associated with causing constipation as a side-effect.

Described herein are compositions and methods for treating constipation that are beneficial in that, for example, they improve the experience of the individual ingesting the compositions and are configured to prevent spoilage and degradation of the laxative within the composition.

### Improved Experience

Traditional laxative based therapies for constipation do not provide a palatable, convenient, appealing or easily portable method for treating constipation. As such, traditional laxative based therapies do not provide a mechanism for encouraging individual compliance with the treatment.

Traditional constipation remedies require ingestion of a laxative along with water. That is, in traditional remedies, a laxative must be dissolved in water or other liquid or be ingested together with ingestion of a liquid. Ingesting a laxative in solution or with the drinking of water, as required with traditional compositions, is inconvenient for the user who must have liquid and a container (for the liquid) available whenever ingesting the traditional laxative composition. In addition, in some cases, an individual must go through the process of measuring out the laxative (e.g. in powder form) and liquid quantities when mixing dry laxative with a liquid to a specific concentration which is an extra, burdensome, and time consuming step that tends to decrease compliance with a regular treatment regimen for constipation.

In contrast, the palatability of the instant compositions combined with the relative convenience that they provide as compared to the traditional treatment method promotes the benefit of improved compliance. That is, because the instant compositions do not require: (1) drinking water with their intake (as traditional formulation do) and (2) do not require mixing with water or other liquid (as some traditional formulations do, one does not need to have water with them or measuring implements (when exact mixing is required), but can rather easily carry and consume the composition wherever is convenient for them without needing to carry out any additional preparation. Which is to say, the instant compositions have increased portability (as compared to traditional formulations) along with providing ease of use (as compared with traditional compositions).

Described herein are compositions and methods that overcome shortcomings in traditional therapies by providing a delivery vehicle for one or more laxatives that provides optimal delivery of the one or more laxatives and/or other active ingredients to the GI tract in order to optimally treat constipation, and further providing a satisfying experience for the individual ingesting the composition thus promoting compliance with the treatment. In some embodiments, the delivery vehicle is a food based delivery vehicle containing a concentrated dose of laxative and does not further require dilution in liquid or concurrent ingestion of liquid. It is notable that when using an osmotic laxative such as polyethylene glycol (PEG), it is counterintuitive to not take the osmotic laxative together with water, the common wisdom being that an osmotic laxative works best when ample water is ingested. In contrast, it is unexpected that osmotic laxatives such as the PEG found in some of the embodiments described herein are effective without the ingestion of water together with the ingestion of the laxative. In addition, as stated, the results of the counterintuitive use of osmotic laxatives is improved patient compliance due to at least improved portability and

### Prevention of Spoilage and Degradation

Described herein are compositions for treating constipation that contain substantially no liquid or very little liquid. Having low amounts of liquid within the composition is beneficial in that it prevents the ingredients of the composition (especially the laxative) from degrading over time during the period from when the composition is manufactured until when it is consumed. It is well known, that compositions having lower Aw (such as the instant compositions) have longer shelf life and are less prone to spoilage (such as bacterial and mold contamination) as well as less prone to chemical degradation (such as through hydrolysis). As such, the instant compositions, which contain relatively little water are expected to deliver a concentrated dose of laxative (relative to the overall size of the composition) and have longer shelf life and better stability than traditional edible compositions that contain water.

### Compositions and Methods

Described herein are compositions and methods for treating constipation in an individual. In some embodiments, the composition and method for treating constipation comprises providing an individual with a composition that comprises a food item comprising one or more ingredients and a concentrated amount of laxative.

Described herein is a method for treating constipation in an individual comprising: providing to the individual a formulation comprising a solid food item comprising PEG mixed together with one or more food ingredients; and instructing the individual to not drink any liquid while ingesting the solid food item. In some embodiments, the solid food item comprises a food bar. In some embodiments, the food bar comprises an edible coating and the PEG is entirely within the edible coating. In some embodiments, the food bar comprises a homogenous mixture of the one or more food ingredients and PEG. In some embodiments, the PEG comprises PEG 3350. In some embodiments, the solid food item comprises 8.5 g of the PEG 3350. In some embodiments, the method comprises ingesting, by the individual, an additional solid food item comprising 8.5 grams of PEG 3350 following a period of time after the individual has ingested the solid food item. In some embodiments, the period of time comprises 6 hours. In some embodiments, the period of time comprises 8 hours. In some embodiments, the period of time comprises 12 hours. In some embodiments, the one or more food ingredients comprise a USP grade food ingredient. In some embodiments, the PEG comprises a powder. In some embodiments, the solid food item comprises 50% or more PEG by weight. In some embodiments, the solid food item comprises 10% or less water by weight. In some embodiments, the solid food item comprises 5% or less water by weight. In some embodiments, the solid food item comprises 1% or less water by weight. In some embodiments, the solid food item comprises 0.5% or less water by weight. In some embodiments, the solid food item comprises a homogenous mixture. In some embodiments, the individual does not drink any liquid within two minutes after having ingested the solid food item. In some embodiments, the constipation comprises chronic idiopathic constipation.

Described herein is a composition for treating constipation comprising: a solid food item comprising one or more food ingredients and PEG; and wherein the composition comprises less than 10% water by weight. In some embodiments, the solid food item comprises a food bar. In some embodiments, the food bar comprises an edible coating and the PEG is entirely within the edible coating. In some embodiments, the PEG comprises a bar interior that is coated by one or more food ingredients. In some embodiments, the food bar comprises a homogenous mixture of the one or more food ingredients and PEG. In some embodiments, the PEG comprises PEG 3350. In some embodiments, the solid food item comprises 45% or more PEG by weight. In some embodiments, the solid food item comprises 8.5 g of the PEG 3350. In some embodiments, the composition comprises 10 grams of fiber or more. In some embodiments, the one or more food ingredients comprise a USP grade food ingredient. In some embodiments, the PEG comprises a powder. In some embodiments, the solid food item comprises 50% or more PEG by weight. In some embodiments, the solid food item comprises 5% or less water by weight. In some embodiments, the solid food item comprises 1% or less water by weight. In some embodiments, the solid food item comprises 0.5% or less water by weight.

### DETAILED DESCRIPTION

Described herein are compositions and methods for treating constipation in an individual. The compositions and methods described herein are effective in treating many types of constipation including the following non-limiting embodiments.

### Constipation Types Treated

Embodiments of the compositions and methods described herein are suitable for treating individuals with constipation types having one or more of the following etiologies: In some embodiments, the constipation treated by the compositions and methods described herein is caused by a disease process such as, for example, Irritable Bowel Syndrome. In some embodiments, the constipation treated by the compositions and methods described herein is caused by a disease process such as an anxiety disorder. In some embodiments, the constipation treated by the compositions and methods described herein is caused by aging. In some embodiments, the constipation treated by the compositions and methods described herein is caused by a wasting disease such as certain cancers. In some embodiments, the constipation treated by the compositions and methods described herein is caused by poor nutrition. In some embodiments, the constipation treated by the compositions and methods described herein is caused by a medication. In some embodiments, the constipation treated by the compositions and methods described herein is caused by an opioid. In some embodiments, the constipation treated by the compositions and methods described herein is caused by codeine. In some embodiments, the constipation treated by the compositions and methods described herein is caused by oxycodone. In some embodiments, the constipation treated by the compositions and methods described herein is caused by hydromorphone. In some embodiments, the constipation treated by the compositions and methods described herein is caused by buprenoprhine. In some embodiments, the constipation treated by the compositions and methods described herein is caused by fentanyl. In some embodiments, the constipation treated by the compositions and methods described herein is caused by hydrocodone. In some embodiments, the constipation treated by the compositions and methods described herein is caused by meperidine. In some embodiments, the constipation treated by the compositions and methods described herein is caused by methadone. In some embodiments, the constipation treated by the compositions and methods described herein is caused by morphine sulfate. In some embodiments, the constipation treated by the compositions and methods described herein is caused by oxymorphone. In some embodiments, the constipation treated by the compositions and methods described herein is caused by tramadol. In some embodiments, the constipation treated by the compositions and methods described herein is caused by an antidepressant. In some embodiments, the constipation treated by the compositions and methods described herein is caused by amitriptyline. In some embodiments, the constipation treated by the compositions and methods described herein is caused by imipramine. In some embodiments, the constipation treated by the compositions and methods described herein is caused by doxepin. In some embodiments, the constipation treated by the compositions and methods described herein is caused by an antihypertensive. In some embodiments, the constipation treated by the compositions and methods described herein is caused by clonidine. In some embodiments, the constipation treated by the compositions and methods described herein is caused by a beta blocker. In some embodiments, the constipation treated by the compositions and methods described herein is caused by atenolol. In some embodiments, the constipation treated by the compositions and methods described herein is caused by an anti-Parkinson's agent. In some embodiments, the constipation treated by the compositions and methods described herein is caused by bromocriptine. In some embodiments, the constipation treated by the compositions and methods described herein is caused by a cholesterol lowering agent. In some embodiments, the constipation treated by the compositions and methods described herein is caused by cholestyramine. In some embodiments, the constipation treated by the compositions and methods described herein is caused by a medication for treatment of gastrointestinal ulcers. In some embodiments, the constipation treated by the compositions and methods described herein is caused by sucralfate. In some embodiments, the constipation treated by the compositions and methods described herein is caused by an anticonvulsant. In some embodiments, the constipation treated by the compositions and methods described herein is caused by phenytoin. In some embodiments, the constipation treated by the compositions and methods described herein is caused by carbamazapine. In some embodiments, the constipation treated by the compositions and methods described herein is caused by an iron supplement. In some embodiments, the constipation treated by the compositions and methods described herein is caused by a calcium channel blocker. In some embodiments, the constipation treated by the compositions and methods described herein is caused by diltiazem. In some embodiments, the constipation treated by the compositions and methods described herein is caused by nifedipine. In some embodiments, the constipation treated by the compositions and methods described herein is caused by an NSAID. In some embodiments, the constipation treated by the compositions and methods described herein is caused by ibuprofen. In some embodiments, the constipation treated by the compositions and methods described herein is caused by an anticholinergic. In some embodiments, the constipation treated by the compositions and methods described herein is caused by diphenhydramine. In some embodiments, the constipation treated by the compositions and methods described herein is caused by cetirizine. In some embodiments, the constipation treated by the compositions and methods described herein is caused by fexofenadine.

### Compositions for Treating Constipation

Generally, the compositions described herein include a delivery vehicle and a solid-form concentrated laxative. A delivery vehicle, as described herein, comprises an edible solid item such as a solid food item. It is also contemplated, however, that a delivery vehicle comprises a tablet, capsule, or other formulation of a solid laxative.

A solid food item, in some embodiments, comprises a discrete food item. In some embodiments, a solid food item comprises a plurality of food items which may comprise elements of a meal. Non-limiting examples of solid food items include food bars, baked goods, meat products, fruit and fruit products, vegetables and plant based products, candies, gums, nuts and nut products.

In some embodiments, the solid food item comprises food ingredients that may comprise any edible food ingredients used to make a food item. Non-limiting examples of ingredients include sugars and natural sweeteners, artificial sweeteners, grain based flour, non-grain based flour, fruit and fruit products, vegetables and vegetable products, dairy products including milk, cream, cheese, and butter, edible oils, chocolate, and nuts.

In some embodiments, one or more ingredients comply with the standards of the National Formulary of the U.S. Pharmacopeial Convention (USP/NF). In some embodiments, a USP/NF grade food ingredient comprises a sweetener. Non-limiting examples of USP/NF grade sweeteners include syrup, invert syrup, erythritol, maltodextrin, and dextrin. In some embodiments, a USP/NF grade ingredient comprises a texturizer. Other non-limiting examples USP/NF grade food ingredients suitable for use with the compositions and methods described herein include cocoa butter, malic acid, citric acid, lemon oil, and vanilla flavor. In some embodiments, the laxative comprises a USP/NF grade laxative. Compositions described herein containing USP/NF grade foods and laxatives are beneficial in that, for example, such compositions provide for improved mouthfeel. The use of USP/NF food ingredients in the compositions described herein is also beneficial in that, for example, it provides the ability to include higher amounts of laxative in said compositions than would be possible if non-USP/NF ingredients were used.

The combination of USP/NF ingredients with very low water or other liquids, in some embodiments of the compositions, is beneficial as a combination in that the very low or absent water content prevents bacterial (or other microbial) contamination and therefore works synergistically with the USP/NF ingredients to provide a highly pure (i.e. uncontaminated) composition.

In some embodiments of the compositions described herein, a composition comprises a solid food item comprising one or more food ingredients combined with one or more laxatives. Non-limiting examples of laxatives suitable for combination with the one or more food ingredients include dibasic sodium phosphate, magnesium citrate, magnesium hydroxide (milk of magnesia), magnesium sulfate (Epsom salt), monobasic sodium phosphate, sodium biphosphate, lactulose, polyethylene glycol (PEG) (including, for example, PEG 3350, PEG 4000, PEG 6000, and PEG 8000), vitamin C, dioctyl sulfosuccinate (Docusate), bisacodyl (Dulcolax), castor oil, lactitol, and sorbitol.

In the compositions described herein that comprise a solid food item, one or more food ingredients are combined with one or more laxatives. In some embodiments, one or more food ingredients are mixed together with one or more laxatives to form the food item. For example, in some embodiments, the solid food item comprises a food bar having one or more laxatives incorporated therein. In some embodiments, the one or more food ingredients and the one or more laxatives form a homogenous mixture. In some embodiments, the one or more food ingredients and one or more laxatives do not comprise a homogenous mixture. In some embodiments, one or more laxatives comprise one or more agglomerated collections of laxative that are at least partially surrounded by one or more food ingredients. For example, in some embodiments, a single agglomerated quantity of PEG 3350 is surrounded by one or more food ingredients of a baked good such as a brownie so that the agglomerated quantity of PEG 3350 is incorporated within the brownie which comprises the food item. In some embodiments, one or more laxatives comprises a coating of an agglomerated quantity of food ingredients. For example, in some embodiments, a food item comprises a bar with a coating comprising of one or more laxatives. In some embodiments, a coating comprises an icing, frosting, or fondant.

The laxative, in some embodiments, comprises PEG 3350. In some embodiments, the quantity of PEG 3350 that is combined with one or more food ingredients comprises 17 grams of PEG 3350. In some embodiments, the quantity of PEG 3350 that is combined with one or more food ingredients comprises 16 grams of PEG 3350. In some embodiments, the quantity of PEG 3350 that is combined with one or more food ingredients comprises 15 grams of PEG 3350. In some embodiments, the quantity of PEG 3350 that is combined with one or more food ingredients comprises 14 grams of PEG 3350. In some embodiments, the quantity of PEG 3350 that is combined with one or more food ingredients comprises 13 grams of PEG 3350. In some embodiments, the quantity of PEG 3350 that is combined with one or more food ingredients comprises 12 grams of PEG 3350. In some embodiments, the quantity of PEG 3350 that is combined with one or more food ingredients comprises 11 grams of PEG 3350. In some embodiments, the quantity of PEG 3350 that is combined with one or more food ingredients comprises 10 grams of PEG 3350. In some embodiments, the quantity of PEG 3350 that is combined with one or more food ingredients comprises 9 grams of PEG 3350. In some embodiments, the quantity of PEG 3350 that is combined with one or more food ingredients comprises 8.5 grams of PEG 3350. In some embodiments, the quantity of PEG 3350 that is combined with one or more food ingredients comprises 8 grams of PEG 3350. In some embodiments, the quantity of PEG 3350 that is combined with one or more food ingredients comprises 7 grams of PEG 3350. In some embodiments, the quantity of PEG 3350 that is combined with one or more food ingredients comprises 6 grams of PEG 3350. In some embodiments, the quantity of PEG 3350 that is combined with one or more food ingredients comprises 5 grams of PEG 3350. In some embodiments, the quantity of PEG 3350 that is combined with one or more food ingredients comprises 9 grams of PEG 3350. In some embodiments, the quantity of PEG 3350 that is combined with one or more food ingredients comprises 8 grams of PEG 3350. In some embodiments, the quantity of PEG 3350 that is combined with one or more food ingredients comprises 7 grams of PEG 3350. In some embodiments, the quantity of PEG 3350 that is combined with one or more food ingredients comprises 6 grams of PEG 3350. In some embodiments, the quantity of PEG 3350 that is combined with one or more food ingredients comprises 5 grams of PEG 3350. In some embodiments, the quantity of PEG 3350 that is combined with one or more food ingredients comprises 4 grams of PEG 3350. In some embodiments, the quantity of PEG 3350 that is combined with one or more food ingredients comprises 3 grams of PEG 3350. In some embodiments, the quantity of PEG 3350 that is combined with one or more food ingredients comprises 2 grams of PEG 3350. In some embodiments, the quantity of PEG 3350 that is combined with one or more food ingredients comprises 1 gram of PEG 3350.

Embodiments of the compositions described herein are configured to promote chewing which promotes gastro-intestinal motility. Improved gastro-intestinal motility is beneficial in that, for example, it helps treat GI dysmotility associated with constipation and helps remove the necessity of ingesting water with the laxative composition. For example, the compositions described herein comprise a solid food item which requires chewing to ingest as compared to, for example, a liquid.

In some embodiments, solid compositions as described herein additionally provide fiber which both promotes chewing and thus gastro-intestinal motility and increases gastro-intestinal bulk which promotes pulling of water into the colon and promotes a bowel movement. For example, in some embodiments, the composition comprises 10 g of fiber. For example, in some embodiments, the composition comprises 9 g of fiber. For example, in some embodiments, the composition comprises 8 g of fiber. For example, in some embodiments, the composition comprises 7 g of fiber. For example, in some embodiments, the composition comprises 6 g of fiber. For example, in some embodiments, the composition comprises 5 g of fiber. For example, in some embodiments, the composition comprises 4 g of fiber. For example, in some embodiments, the composition comprises 3 g of fiber. For example, in some embodiments, the composition comprises 2 g of fiber. For example, in some embodiments, the composition comprises 1 g of fiber. In some embodiments, one or more compositions described herein either individually or in combination provide 25 g to 35 g of fiber per day to an individual when consumed in a day.

Fiber incorporated into some of the embodiments described herein is 100% water soluble. In some embodiments, fiber as used herein is 95% water soluble. In some embodiments, fiber as used herein is 90% water soluble. In some embodiments, fiber as used herein is 85% water soluble. In some embodiments, fiber as used herein is 80% water soluble. In some embodiments, fiber as used herein is 75% water soluble. In some embodiments, fiber as used herein is 70% water soluble. In some embodiments, fiber as used herein is 65% water soluble. In some embodiments, fiber as used herein is 65% water soluble. In some embodiments, fiber as used herein is 60% water soluble. In some embodiments, fiber as used herein is 55% water soluble. In some embodiments, fiber as used herein is 50% water soluble.

Non-limiting examples of suitable fiber sources include Methylcellulose, Calcium Polycarbophil, Psyllium Husk, and Inulin. In some embodiments, a composition as described herein comprises 2 g of Methylcellulose. In some embodiments, a composition as described herein comprises 1 g of Calcium Polycarbophil. In some embodiments, a composition as described herein comprises 3.4 g of Psyllium Husk. In some embodiments, a composition as described herein comprises 3 g of Inulin.

### High Concentration of Laxatives

The compositions described herein provide a laxative in a concentrated form. Some ways in which a high concentration of laxative is provided within the compositions described herein include, for example: (a) providing a relatively high percent by weight of laxative within the composition, (b) providing one or more laxatives in a solid undissolved form, (c) providing one or more laxatives within a composition having low amount of water or other liquids or absence of water or other liquids, and (d) providing a composition as described for an individual to ingest without concurrently ingesting any liquids.

(a) Providing a Relatively High Concentration of Laxative by Weight

As stated, compositions for treating constipation described herein are formulated to be edible and in some embodiments comprise a solid food item containing a laxative. The laxative is concentrated with respect to the composition by, for example, providing a relatively high percentage of laxative by weight relative to the overall weight of the composition. Parameters that determine what percent by weight of the composition of a laxative include, for example, the type of laxative used, whether additional laxatives are provided within the composition, and what the routine dosing is of the laxative for treating constipation. For example, in some embodiments, a composition as described herein comprises 30% laxative by weight. For example, in some embodiments, a composition as described herein comprises 35% laxative by weight. For example, in some embodiments, a composition as described herein comprises 40% laxative by weight. For example, in some embodiments, a composition as described herein comprises 45% laxative by weight. For example, in some embodiments, a composition as described herein comprises 50% laxative by weight. For example, in some embodiments, a composition as described herein comprises 55% laxative by weight. For example, in some embodiments, a composition as described herein comprises 60% laxative by weight. For example, in some embodiments, a composition as described herein comprises 65% laxative by weight. For example, in some embodiments, a composition as described herein comprises 70% laxative by weight. For example, in some embodiments, a composition as described herein comprises 75% laxative by weight. For example, in some embodiments, a composition as described herein comprises 80% laxative by weight. For example, in some embodiments, a composition as described herein comprises 85% laxative by weight. For example, in some embodiments, a composition as described herein comprises 90% laxative by weight. For example, in some embodiments, a composition as described herein comprises 95% laxative by weight.

In some embodiments, a single laxative is used in the composition comprising PEG 3350 and the percentage by weight of the PEG 3350 is greater than 50% (i.e. greater than 50% of the total weight of the composition).

Table 1 below shows a formulation for an exemplary composition for treating constipation comprising a lemon bar that comprises PEG 3350.

TABLE 1
Lemon Bar

Amount per Unit (grams) 24.4

| Ingredient | % by weight | |
|---|---|---|
| Polyethylene Glycol 3350 | 69.8% | 17.023 |
| Invert Syrup | 17.0% | 4.142 |
| Cocoa Butter | 12.4% | 3.018 |
| Flavor, Milky Vanilla N&A | 0.7% | 0.171 |
| #2713, David Michael | | |
| Citric Acid | 0.1% | 0.029 |
| Lemon Oil | 0.1% | 0.016 |
| | 100.0% | 24.400 |

As shown in table 1, a lemon bar comprises 69.8% by weight PEG 3350 with a total amount of PEG of 17.023 grams. One or more food ingredients include invert syrup, cocoa butter, vanilla, citric acid, and lemon oil. In some embodiments, one or more ingredients are USP-NF grade

Table 2 below shows a formulation for an exemplary composition for treating constipation comprising a white chocolate bar that comprises PEG 3350.

TABLE 2
White Chocolate Bar

Amount per Unit (grams) 24.4

| Ingredient | % by weight | |
|---|---|---|
| Polyethylene Glycol 3350 | 69.767% | 17.023 |
| Invert Syrup | 16.772% | 4.092 |
| Cocoa Butter | 11.860% | 2.894 |
| Flavor, Milky Vanilla N&A | 0.700% | 0.171 |
| #2713, David Michael | | |
| Flavor, Chocolate N&A | 0.800% | 0.195 |
| #50083, David Michael | | |
| Flavor, Creamy Vanilla O.S. | 0.100% | 0.024 |
| #50043, David Michael | | |
| | 100.000% | 24.400 |

As shown in table 2, a white chocolate bar comprises 69.767% by weight PEG 3350 with a total amount of PEG of 17.023 grams. One or more food ingredients include invert syrup, cocoa butter, vanilla, and chocolate flavor. In some embodiments, one or more ingredients are USP-NF grade.

Table 3 below shows a formulation for an exemplary composition for treating constipation comprising a coconut bar that comprises PEG 3350.

TABLE 3
Coconut Bar

Amount per Unit (grams) 24.4

| Ingredient | % by weight | |
|---|---|---|
| Polyethylene Glycol 3350 | 69.767% | 17.023 |
| Invert Syrup | 16.915% | 4.127 |
| Cocoa Butter | 12.267% | 2.993 |
| Flavor, Coconut | 0.800% | 0.195 |
| #3728, David Michael | | |
| Flavor, Creamy Vanilla O.S. | 0.250% | 0.061 |
| #50043, David Michael | | |
| | 100.000% | 24.400 |

As shown in table 3, a coconut bar comprises 69.767% by weight PEG 3350 with a total amount of PEG of 17.023 grams. One or more food ingredients include invert syrup, cocoa butter, vanilla, and coconut flavor. In some embodiments, one or more ingredients are USP-NF grade.

Table 4 below shows a formulation for an exemplary composition for treating constipation comprising a chocolate mint bar that comprises PEG 3350.

TABLE 4
Chocolate Mint

Amount per Unit (grams)

| Ingredient | % by weight | |
|---|---|---|
| Polyethylene Glycol 3350 Powder | 50.50% | 8.500 |
| Chocolate Barry Callebaut | 39.30% | 6.610 |
| CHD DR 604510-017 | | |
| Sunflower Oil AAK SUN 46-53 | 10.00% | 1.680 |
| Flavor, Peppermint | 0.20% | 0.030 |
| #51365, David Michael | | |
| | 100.00% | 16.820 |

As shown in table 4, a chocolate mint bar comprises 50.50% by weight PEG 3350 with a total amount of PEG of 8.5 grams. One or more food ingredients include chocolate, peppermint flavor, and sunflower oil. In some embodiments, one or more ingredients are USP-NF grade.

Table 5 below shows a formulation for an exemplary composition for treating constipation comprising a salted caramel bar that comprises PEG 3350.

TABLE 5
Salted Caramel

Amount per Unit (grams)

| Ingredient | % by weight | |
|---|---|---|
| Polyethylene Glycol 3350 Powder | 50.00% | 8.500 |
| Cocoa Butter | 22.35% | 3.830 |
| Sugar - Domino Powder | 18.40% | 3.130 |
| Sunflower Oil AAK SUN 46-53 | 7.50% | 1.280 |
| Flavor, Toffee UA 3852, Ungerer | 1.00% | 0.170 |
| Flavor, Vanilla UA9088, Ungerer | 0.30% | 0.050 |
| salt | 0.30% | 0.000 |
| color | 0.15% | |
| | 100.00% | 16.960 |

As shown in table 5, a salted caramel bar comprises 50.00% by weight PEG 3350 with a total amount of PEG of 8.5 grams. One or more food ingredients include cocoa butter, vanilla flavor, and sunflower oil. In some embodiments, one or more ingredients are USP-NF grade.

Table 6 below shows a formulation for an exemplary composition for treating constipation comprising a herbal creme bar that comprises PEG 3350.

TABLE 6
Herbal Crème

Amount per Unit (grams)

| Ingredient | % by weight | |
|---|---|---|
| Polyethylene Glycol 3350 Powder | 50.00% | 8.500 |
| Cocoa Butter | 22.50% | 3.830 |
| Sugar - Domino Powder | 18.50% | 3.150 |
| Sunflower Oil AAK SUN 46-53 | 7.50% | 1.280 |
| Flavor, Chamomile UA9086, Ungerer | 0.60% | 0.100 |
| Flavor, Tea UA9087, Ungerer | 0.50% | 0.090 |
| Flavor, Vanilla UA9088, Ungerer | 0.40% | 0.070 |
| | 100.00% | 17.020 |

As shown in table 6, a herbal creme bar comprises 50.00% by weight PEG 3350 with a total amount of PEG of 8.5 grams. One or more food ingredients include cocoa butter, vanilla flavor, chamomile, and sunflower oil. In some embodiments, one or more ingredients are USP-NF grade.

Table 7 below shows a formulation for an exemplary composition for treating constipation comprising a lemon creme bar that comprises PEG 3350.

TABLE 7
Lemon Crème

Amount per Unit (grams)

| Ingredient | % by weight | |
|---|---|---|
| Polyethylene Glycol 3350 Powder | 50.40% | 8.500 |
| Cocoa Butter | 22.70% | 3.830 |
| Sugar - Domino Powder | 18.50% | 3.150 |
| Sunflower Oil AAK SUN 46-53 | 7.50% | 1.280 |
| Flavor, Milky Vanilla N&A | 0.70% | 0.100 |
| #2713, David Michael | | |
| Citric Acid | 0.12% | 0.090 |
| Lemon Oil | 0.07% | 0.070 |
| Color, Yellow | 0.01% | 0.070 |
| #5 Lake 8-10%, Sensient | | |
| | 100.00% | 17.090 |

As shown in table 7, a herbal creme bar comprises 50.40% by weight PEG 3350 with a total amount of PEG of 8.5 grams. One or more food ingredients include cocoa butter, vanilla flavor, lemon oil, and sunflower oil. In some embodiments, one or more ingredients are USP-NF grade.

In the examples shown in tables 1-3, the total weight of PEG 3350 is around 17 g and comprises around 70% by weight of the total bar weight. In the examples shown in tables 4-7, the total weight of PEG 3350 is around 8.5 g and comprises around 50% by weight of the total bar weight. In other embodiments, PEG 3350 has other total weights within the compositions described herein. For example, in some embodiments, the total PEG 3350 comprises 16 g. For example, in some embodiments, the total PEG 3350 comprises 15 g. For example, in some embodiments, the total PEG 3350 comprises 14 g. For example, in some embodiments, the total PEG 3350 comprises 13 g. For example, in some embodiments, the total PEG 3350 comprises 12 g. For example, in some embodiments, the total PEG 3350 comprises 11 g. For example, in some embodiments, the total PEG 3350 comprises 10 g. For example, in some embodiments, the total PEG 3350 comprises 9.5 g. For example, in some embodiments, the total PEG 3350 comprises 8.5 g. For example, in some embodiments, the total PEG 3350 comprises 8.0 g. For example, in some embodiments, the total PEG 3350 comprises 7.5 g. For example, in some embodiments, the total PEG 3350 comprises 7.0 g. For example, in some embodiments, the total PEG 3350 comprises 6.5 g. For example, in some embodiments, the total PEG 3350 comprises 6.0 g. For example, in some embodiments, the total PEG 3350 comprises 5.5 g. For example, in some embodiments, the total PEG 3350 comprises 5 g. For example, in some embodiments, the total PEG 3350 comprises 4.5 g. For example, in some embodiments, the total PEG 3350 comprises 4 g. For example, in some embodiments, the total PEG 3350 comprises 3.5 g. For example, in some embodiments, the total PEG 3350 comprises 3 g. For example, in some embodiments, the total PEG 3350 comprises 2.5 g. For example, in some embodiments, the total PEG 3350 comprises 2 g. For example, in some embodiments, the total PEG 3350 comprises 1.5 g. For example, in some embodiments, the total PEG 3350 comprises 1 g.

Embodiments having relatively smaller doses of laxatives allow for modular dosing therapy. For example, where the total daily recommended dose of PEG 3350 is 17 g, to food items each containing 8.5 g of PEG 3350 may be provided to an individual to treat constipation. Where in this example, a first food item (containing 8.5 g of PEG 3350) is ingested in the morning and a second food item (also containing 8.5 g of PEG 3350) is ingested at night. In this way, the total dosage of PEG 3350 is administered over time rather than in one single dose which provides more effective therapy for some individuals. It should be understood that numerous combinations of dosages may be utilized with the compositions and methods described herein. For example, if the total recommended daily dose of PEG 3350 is 17 g, a first composition ingested may contain 10 g of PEG 3350 and a second may contain 7 g of PEG 3350. Similarly, the total daily dose of laxative may be divided over more than two separate compositions, for instance, over three or more doses. In this way, modular dosing of PEG 3350 using different doses within different compositions allows optimization and personalization of therapy. It should also be understood that multiple doses are configured, in some embodiments, to be ingested concurrently. For example, an individual ingests a two food items each containing 8.5 g of PEG 3350 at the same time or essentially the same time to deliver a total daily dose of 17 g of PEG. In some embodiments, an individual consumes the two food items in separate episodes over the course of 24 hours. In some embodiments, an individual ingests more than two food items over the course of 24 hours for a total PEG dose of more than 17 g per day.

As indicated above, PEG 3350 comprised around 70% by weight of the entire composition in each of the compositions of tables 1-3 and PEG 3350 comprised around 50% by weight of the entire composition in each of the compositions of tables 4-7. In this way, PEG 3350 is provided in a solid form that is concentrated relative to the total weight of the composition.

It should also be noted, that as described above, in some embodiments, two or more different laxatives are incorporated within a single composition. For example, in an embodiment, lactitol and PEG 3350 are combined within a single composition comprising a single food item. For example, in one embodiment a solid food item comprises 15 ml of lactitol and 8.5 g of PEG 3350 mixed together with one or more food ingredients.

(b) Providing One or More Laxatives in a Solid Undissolved Form

In traditional formulations of certain laxatives including PEG 3350, the laxative is dissolved in water or another liquid and as such is delivered in a diluted form. In contrast, in embodiments of the instant compositions and methods, laxative is provided in a concentrated solid and undissolved form so that the laxative is not diluted by liquid.

Laxative in a solid (or undiluted liquid form) is mixed directly with one or more food ingredients to form a solid food item. For example, PEG 3350 in powder form is mixed directly with one or more dry food ingredients to form a food bar in an embodiment of the compositions described herein. In these embodiments, the dry food ingredients and powdered laxative are formed into a bar using one or more binders.

(c) Providing One or More Laxatives within a Composition Having a Very Low Amount of Water or Other Liquids or Absence of Water or Other Liquids

Further, in embodiments, either no water or other liquid or minimal water or other liquid is added to the composition in order to concentrate the laxative. For example, in some embodiments, the composition is 15% or less water or other liquid by weight. For example, in some embodiments, the composition is 14% or less water or other liquid by weight. For example, in some embodiments, the composition is 13% or less water or other liquid by weight. For example, in some embodiments, the composition is 12% or less water or other liquid by weight. For example, in some embodiments, the composition is 10% or less water or other liquid by weight. For example, in some embodiments, the composition is 9% or less water or other liquid by weight. For example, in some embodiments, the composition is 8% or less water or other liquid by weight. For example, in some embodiments, the composition is 7% or less water or other liquid by weight. For example, in some embodiments, the composition is 6% or less water or other liquid by weight. For example, in some embodiments, the composition is 5% or less water or other liquid by weight. For example, in some embodiments, the composition is 4% or less water or other liquid by weight. For example, in some embodiments, the composition is 3% or less water or other liquid by weight. For example, in some embodiments, the composition is 2% or less water or other liquid by weight. For example, in some embodiments, the composition is 1% or less water or other liquid by weight. For example, in some embodiments, the composition comprises 0% water or other liquid by weight.

In some embodiments of the compositions described herein no water is added to the composition. Therefore, any water added to the composition comes primarily from moisture that is inherent in the excipients (e.g. food ingredients) and laxative (e.g. PEG3350). For example, the water content based on Karl Fischer analysis of some excipients used in some embodiments of the compositions described herein is as follows:

Chocolate Mint—0.7%

Lemon Creme—0.4%

Salted Caramel—0.7%

In some embodiments of the compositions described herein, the composition has a water activity of 0.5 or less. In some embodiments of the compositions described herein, the composition has a water activity of 0.6 or less. In some embodiments of the compositions described herein, the composition has a water activity of 0.7 or less. In some embodiments of the compositions described herein, the composition has a water activity of 0.8 or less.

(d) Providing a Composition as Described for an Individual to Ingest without Concurrently Ingesting any Liquids

In some embodiments, a method for treating constipation comprises providing a composition to an individual comprising a solid food item comprising one or more laxatives mixed together with one or more food ingredients. The individual then ingests the composition without concurrently ingesting any fluids. In this manner, no dilution of the composition occurs through the ingestion of liquid concurrently to the ingestion of the composition.

In some embodiments of the method an individual does not ingest any liquid including water within one hour of ingesting the composition. In some embodiments of the method an individual does not ingest any liquid including water within 30 minutes of ingesting the composition. In some embodiments of the method an individual does not ingest any liquid including water within 20 minutes of ingesting the composition. In some embodiments of the method an individual does not ingest any liquid including water within 15 minutes of ingesting the composition. In some embodiments of the method an individual does not ingest any liquid including water within 10 minutes of ingesting the composition. In some embodiments of the method an individual does not ingest any liquid including water within 9 minutes of ingesting the composition. In some embodiments of the method an individual does not ingest any liquid including water within 8 minutes of ingesting the composition. In some embodiments of the method an individual does not ingest any liquid including water within 7 minutes of ingesting the composition. In some embodiments of the method an individual does not ingest any liquid including water within 6 minutes of ingesting the composition. In some embodiments of the method an individual does not ingest any liquid including water within 5 minutes of ingesting the composition. In some embodiments of the method an individual does not ingest any liquid including water within 4 minutes of ingesting the composition. In some embodiments of the method an individual does not ingest any liquid including water within 3 minutes of ingesting the composition. In some embodiments of the method an individual does not ingest any liquid including water within 2 minutes of ingesting the composition. In some embodiments of the method an individual does not ingest any liquid including water within 1 minutes of ingesting the composition. In some embodiments of the method an individual does not ingest any liquid including water within 30 seconds of ingesting the composition.

### Manufacture

Any of the embodiments of the compositions described herein are prepared utilizing a low temperature manufacturing process, so that, in particular, minimal (or no) heat is applied to one or more laxatives within the compositions described herein. It should be understood, that the application of high temperature to a laxative will cause degradation of the laxative decreasing the efficacy of the laxative as well as potentially releasing toxic degradation products. In the instant described manufacturing methods, the efficacy of the laxative is maintained and the release of potentially toxic degradation products is avoided.

An exemplary method of making PEG-based laxative-containing food bars comprises (1) mixing a PEG laxative powder (or other form of PEG) with dry ingredients, for example, sea salt or flavor powders; (2) blending the PEG and dry ingredient mixture with a melted binder and/or glycerin, preferably at room temperature, for example, a temperature around or below 80° F. (26.7° C.), to form a homogenous malleable semi-solid mixture (for example, a fondant-like mass); (3) blending the malleable semi-solid mixture with rice syrup to form a homogeneous dispersion; (4) optionally folding additional ingredients, for example, rice crisps, dried fruit, or chocolate chips, into the dispersion; and (5) forming the dispersion into a bar, for example, of about 35 or 70 grams.

Non-limiting examples of processes for manufacturing a composition comprising a solid food item as described herein include mixing, cooking, and baking one or more food ingredients and/or one or more laxatives. In some embodiments, the one or more food ingredients and the one or more laxatives all coalesce together. When one or more food ingredients and/or one or more laxatives are heated in order to manufacture the food item, the heating is done at a temperature wherein the food ingredients tend to coalesce together (i.e. with each other as well as with one or more laxatives) while the laxatives do not degrade. In addition, typically, heat is not applied around a temperature at which a therapeutic property of one or more laxatives of the compound are affected. The effect of heating is determined by both the temperature and duration over which heat is applied. In some embodiments, a compound for treating constipation comprises a mixture of one or more laxatives and one or more food ingredients which are heated to a temperature around 350 degrees Fahrenheit. In some embodiments, a compound for treating constipation comprises a mixture of one or more laxatives and one or more food ingredients which are heated to a temperature around 300 degrees Fahrenheit. In some embodiments, a compound for treating constipation comprises a mixture of one or more laxatives and one or more food ingredients which are heated to a temperature around 250 degrees Fahrenheit. In some embodiments, a compound for treating constipation comprises a mixture of one or more laxatives and one or more food ingredients which are heated to a temperature around 200 degrees Fahrenheit. In some embodiments, a compound for treating constipation comprises a mixture of one or more laxatives and one or more food ingredients which are heated to a temperature around 150 degrees Fahrenheit. In some embodiments, a compound for treating constipation comprises a mixture of one or more laxatives and one or more food ingredients which are heated to a temperature around 100 degrees Fahrenheit. In some embodiments, a compound for treating constipation comprises a mixture of one or more laxatives and one or more food ingredients which are heated to a temperature around 95 degrees Fahrenheit. In some embodiments, a compound for treating constipation comprises a mixture of one or more laxatives and one or more food ingredients which are heated to a temperature around 90 degrees Fahrenheit. In some embodiments, a compound for treating constipation comprises a mixture of one or more laxatives and one or more food ingredients which are heated to a temperature around 85 degrees Fahrenheit. In some embodiments, a compound for treating constipation comprises a mixture of one or more laxatives and one or more food ingredients which are heated to a temperature around 80 degrees Fahrenheit. In some embodiments, a compound for treating constipation comprises a mixture of one or more laxatives and one or more food ingredients which are heated to a temperature around 75 degrees Fahrenheit.

In some embodiments, the duration of heating is 1 hour or greater. In some embodiments, the duration of heating is 45 minutes or greater. In some embodiments, the duration of heating is 30 minutes or greater. In some embodiments, the duration of heating is 20 minutes or greater. In some embodiments, the duration of heating is 15 minutes or greater. In some embodiments, the duration of heating is 10 minutes or greater. In some embodiments, the duration of heating is 5 minutes or greater. In some embodiments, the duration of heating is 1 minute or greater.

In some embodiments, a compound for treating constipation further comprises a binder, wherein a binder is an element used to bind one or more elements of the compound together. In some embodiments, the binder is edible. In some embodiments, the binder comprises cocoa butter. In some embodiments, the binder comprises coconut oil. In some embodiments, the binder comprises cocoa butter. In some embodiments, the binder comprises sunflower seed oil. In some embodiments, a binder is heated and the elements of the compound are added to the heated binder. In some embodiments, one or more elements of the compound are added to the binder at the hottest temperature to which it is heated. In some embodiments, one or more elements of the compound are added to the heated binder after it has cooled. In some embodiments, the binder is a liquid when heated so that when mixed with the elements of the compound, the elements coalesce or are “bound” together by the binder. In an exemplary embodiment, a binder in the compound is in a solid state at room temperature and a liquid state at a relatively small increase in temperature above room temperature. This exemplary binder is used in binding the elements of the compound when a solid compound is used such as, for example, a bar. In the liquid state the binder coalesces the elements, and, as stated, the exemplary binder is in a liquid state at temperatures slightly above room temperature, thus the other elements of the compound are not heated due to the binder to a large extent which is especially important with respect to the laxatives that are damaged at high temperatures. In some embodiments, a binder is heated to a temperature around 95 degrees Fahrenheit and then one or more elements of the compound for treating constipation are mixed together with it. In some embodiments, a binder is heated to a temperature around 90 degrees Fahrenheit and then one or more elements of the compound for treating constipation are mixed together with it. In some embodiments, a binder is heated to a temperature around 85 degrees Fahrenheit and then one or more elements of the compound for treating constipation are mixed together with it. In some embodiments, a binder is heated to a temperature around 80 degrees Fahrenheit and then one or more elements of the compound for treating constipation are mixed together with it. In some embodiments, a binder is heated to a temperature around 75 degrees Fahrenheit and then one or more elements of the compound for treating constipation are mixed together with it. In some embodiments, a binder is heated to a temperature around 70 degrees Fahrenheit and then one or more elements of the compound for treating constipation are mixed together with it. In some embodiments, after being heated to an initial temperature, the binder is allowed to cool 30 degrees from the initial temperature before one or more elements of the compound for treating constipation are added. In some embodiments, after being heated to an initial temperature, the binder is allowed to cool 25 degrees from the initial temperature before one or more elements of the compound for treating constipation are added. In some embodiments, after being heated to an initial temperature, the binder is allowed to cool 20 degrees from the initial temperature before one or more elements of the compound for treating constipation are added. In some embodiments, after being heated to an initial temperature, the binder is allowed to cool 15 degrees from the initial temperature before one or more elements of the compound for treating constipation are added. In some embodiments, after being heated to an initial temperature, the binder is allowed to cool 10 degrees from the initial temperature before one or more elements of the compound for treating constipation are added. In some embodiments, after being heated to an initial temperature, the binder is allowed to cool 5 degrees from the initial temperature before one or more elements of the compound for treating constipation are added. It is understood that numerous other edible binders are suited, non-limiting examples of which include lard, vegetable shortening, palm oil, butter, or margarine.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for treating constipation in an individual comprising:
providing to the individual, a formulation comprising a solid food item comprising polyethylene glycol (PEG) mixed together with one or more food ingredients; and
instructing the individual to not drink any liquid while ingesting the solid food item.

2. The method of claim 1, wherein the solid food item comprises a food bar.

3. The method of claim 2, wherein the food bar comprises an edible coating and the PEG is entirely within the edible coating.

4. The method of claim 2, wherein the food bar comprises a homogenous mixture of the one or more food ingredients and PEG.

5. The method of claim 1, wherein the PEG comprises PEG 3350.

6. The method of claim 5, wherein the solid food item comprises 8.5 g of the PEG 3350.

7. The method of claim 6, comprising ingesting, by the individual, an additional solid food item comprising 8.5 grams of PEG 3350 following a period of time after the individual has ingested the solid food item.

8. The method of claim 7, wherein the period of time comprises 6 hours.

9. The method of claim 7, wherein the period of time comprises 8 hours.

10. The method of claim 7, wherein the period of time comprises 12 hours.

11. The method of claim 1, wherein the one or more food ingredients comprise a USP grade food ingredient.

12. The method of claim 1, wherein the PEG comprises a powder.

13. The method of claim 1, wherein the solid food item comprises 50% or more PEG by weight.

14. The method of claim 1, wherein the solid food item comprises 10% or less water by weight.

15. The method of claim 1, wherein the solid food item comprises 5% or less water by weight.

16. The method of claim 1, wherein the solid food item comprises 1% or less water by weight.

17. The method of claim 1, wherein the solid food item comprises 0.5% or less water by weight.

18. The method of claim 1, wherein the solid food item comprises a homogenous mixture.

19. The method of claim 1, comprising instructing the individual further to not drink any liquid within two minutes after having ingested the solid food item.

20. The method of claim 1, wherein the constipation comprises chronic idiopathic constipation.

21. A composition for treating constipation comprising:
a solid food item comprising one or more food ingredients and PEG; and
wherein the composition comprises less than 10% water by weight.

22. The composition of claim 21, wherein the solid food item comprises a food bar.

23. The composition of claim 22, wherein the food bar comprises an edible coating and the PEG is entirely within the edible coating.

24. The composition of claim 22, wherein the PEG comprises a bar interior that is coated by one or more food ingredients.

25. The composition of claim 22, wherein the food bar comprises a homogenous mixture of the one or more food ingredients and PEG.

26. The composition of claim 21, wherein the PEG comprises PEG 3350.

27. The composition of claim 26, wherein the solid food item comprises 50% or more PEG by weight.

28. The composition of claim 26, wherein the solid food item comprises 8.5 g of the PEG 3350.

29. The composition of claim 21, wherein the composition comprises 10 grams of fiber or more.

30. The composition of claim 21, wherein the one or more food ingredients comprise a USP grade food ingredient.

31. The composition of claim 21, wherein the PEG comprises a powder.

32. The composition of claim 21, wherein the solid food item comprises 45% or more PEG by weight.

33. The composition of claim 21, wherein the solid food item comprises 5% or less water by weight.

34. The composition of claim 21, wherein the solid food item comprises 1% or less water by weight.

35. The composition of claim 21, wherein the solid food item comprises 0.5% or less water by weight.
